(19) United States
(12) Patent Application Publication
(10) Pub. No.: US 2019/0382538 A1
(43) Pub. Date: Dec. 19, 2019

(54) Hydrogel Composition and Method for Producing Same

(71) Applicant: Shimadzu Corporation, Kyoto (JP)

(72) Inventors: Hayato Matsui, Kyoto-shi, Kyoto (JP); Takashi Kawabe, Kyoto-shi, Kyoto (JP)

(73) Assignee: Shimadzu Corporation, Kyoto (JP)

(21) Appl. No.: 16/478,874

(22) PCT Filed: Jan. 19, 2017
(86) PCT No.: PCT/JP2017/001795

§ 371 Date: Jul. 18, 2019

(51) Int. Cl. C08J 3/075; A61K 9/51; C08L 87/00

(52) U.S. Cl. CPC C08J 3/075; A61K 9/5192; A61K 9/5146; C08L 87/005; C08J 2377/00; C08L 2201/06; C08L 2203/02; C08L 2205/14; C08J 2367/04; C08L 2201/54

(57) ABSTRACT

The hydrogel composition of the present invention includes an amphiphilic block polymer having a hydrophilic block chain having 20 or more sarcosine units and a hydrophobic block chain having 10 or more lactic acid units, and water as a dispersion medium. In the hydrogel composition, the amphiphilic block polymer is preferably present as hydrogel nano-particles having a particle diameter of 100 nm or less. The hydrogel can be prepared by mixing an amphiphilic block polymer with an aqueous liquid. The hydrogel is preferably substantially free of an organic solvent.

### TECHNICAL FIELD

The present invention relates to a hydrogel composition and a method for producing the same.

### BACKGROUND ART

Biodegradable polymer gels are used in the fields of medical care, foods, cosmetics, and the like. For example, Patent Document 1 discloses a gel composition for subcutaneous injection in which a lactic acid-glycolic acid copolymer (PLGA) is dissolved in a water-soluble solvent such as N-methyl pyrrolidone (NMP). When the gel composition is introduced into a living body by subcutaneous injection, the solvent is replaced with water in the living body, the polymer is solidified by the moisture, and the composition functions as a depot having sustained drug release properties. Patent Document 2 discloses that a gel composition having sustained drug release properties is obtained by dissolving PLGA in a mixed solvent of a water-insoluble solvent such as ethyl benzoate and a water-soluble solvent such as N-methylpyrrolidone.

Patent Document 3 discloses that a hydrogel having sustained drug release properties is obtained by dispersing a water-soluble drug in an organogel of a biodegradable block polymer, then removing a solvent (dispersion medium) from the organogel to form a xerogel, and swelling the xerogel in an aqueous solution.

### PRIOR ART DOCUMENTS

### Patent Documents

Patent Document 1: WO 90/3768

Patent Document 2: WO 98/27963

Patent Document 3: WO 2013/86015

### SUMMARY OF THE INVENTION

### Problems to be Solved by the Invention

The sustained drug release gel compositions as described above reside in the body for several days to several months after being administered into a living body, and thus may cause inflammation at the administration site. Therefore, in applications such as a carrier substrate for cell transplantation, development of a quick-acting and degradable gel composition that is rapidly degraded after acting as a carrier for delivering a target substance into a living body is required.

In addition, since amphiphilic polymers used as gel materials are generally poorly soluble in water, water-soluble organic solvents (amphiphilic solvents) such as NMP and lower alcohols are often used to prepare gels. These organic solvents may cause inflammation at the administration site. Therefore, there is a need for gel compositions that are substantially free of an organic solvent such as lower alcohols.

In view of the above, an object of the present invention is to provide a gel composition which is preparable without using an organic solvent, and which is rapidly degraded after being introduced into a living body.

### Means for Solving the Problems

The present invention relates to a hydrogel composition and a method for producing the same. The hydrogel composition of the present invention includes an amphiphilic block polymer having a hydrophilic block chain having 20 or more sarcosine units and a hydrophobic block chain having 10 or more lactic acid units, and water as a dispersion medium. In the hydrogel composition, the amphiphilic block polymer is preferably present as hydrogel nano-particles having a particle diameter of 100 nm or less.

Preferably, the hydrogel composition is substantially free of an organic solvent. The content of the organic solvent in the hydrogel composition is preferably 0.1% by weight or less. The content of a lower alcohol in the hydrogel composition is preferably 0.01% by weight or less.

### Effects of the Invention

The hydrogel composition of the present invention loses its gel structure in a short time when injected into water, so its residence time in a living body is short. Also, the hydrogel composition of the present invention can be prepared without using an organic solvent such as a lower alcohol. Therefore, the hydrogel composition of the present invention has a low load on a living body and is suitable for administration to the living body as a carrier substrate or the like for cell transplantation.

### BRIEF DESCRIPTION OF THE DRAWINGS

FIG. 1 is photographs of gels obtained in Experimental Examples (Preparation Examples 1 to 3).

FIG. 2 is a TEM observation image of the hydrogel of Experimental Example 1.

FIG. 3 is a TEM observation image of the hydrogel of Experimental Example 2.

FIG. 4 is photographs showing the state of Experimental Example 2 (injection of the gel into water).

FIG. 5 is photographs showing time-lapse changes after injection of the gel into water.

FIG. 6 is TEM observation images of water injected with the hydrogel of Preparation Example 1.

FIG. 7 is TEM observation images of water injected with the hydrogel of Preparation Example 2.

### MODE FOR CARRYING OUT THE INVENTION

The hydrogel composition of the present invention includes an amphiphilic block polymer having a hydrophilic block chain and a hydrophobic block chain, and water as a dispersion medium.

[Amphiphilic Block Polymer]

The hydrogel composition of the present invention is a composition including an amphiphilic block polymer having a hydrophilic block chain and a hydrophobic block chain as a main component. The hydrophilic block chain of the amphiphilic block polymer has a sarcosine unit as a monomer unit, and the hydrophobic block chain thereof has a lactic acid unit as a monomer unit.

(Hydrophobic Block Chain)

The hydrophobic block chain contains 10 or more lactic acid units. Polylactic acid has excellent biocompatibility and stability. In addition, polylactic acid has excellent biodegradability, and thus is metabolized quickly and has low accumulation in a living body. Therefore, amphiphilic block polymers having polylactic acid as a constituent block are useful in applications to living bodies, particularly to human bodies. Further, since polylactic acid is crystalline, even when the hydrophobic block chain is short, the hydrophobic block chain is likely to aggregate in the presence of the dispersion medium to form a physical gel.

The upper limit of the number of lactic acid units in the hydrophobic block chain is not particularly limited, but the number of lactic acid units is preferably 1000 or less from the viewpoint of stabilizing the structure of the gel in the dispersion medium. The number of lactic acid units in the hydrophobic block is preferably 10 to 1000, more preferably 15 to 500, and still more preferably 20 to 100.

The lactic acid unit constituting the hydrophobic block chain may be L-lactic acid or D-lactic acid. In addition, L-lactic acid and D-lactic acid may be mixed. In the hydrophobic block chain, all the lactic acid units may be continuous, or the lactic acid units may be discontinuous. The monomer unit other than the lactic acid contained in the hydrophobic block chain is not particularly limited, and examples thereof include hydroxy acids such as glycolic acid and hydroxyisobutyric acid, and hydrophobic amino acids or amino acid derivatives such as glycine, alanine, valine, leucine, isoleucine, proline, methionine, tyrosine, tryptophan, glutamic acid methyl ester, glutamic acid benzyl ester, aspartic acid methyl ester, aspartic acid ethyl ester, and aspartic acid benzyl ester.

(Hydrophilic Block Chain)

The hydrophilic block chain contains 20 or more sarcosine units (N-methylglycine units). Sarcosine is highly water soluble. In addition, polysarcosine is capable of cis-trans isomerization because it has an N-substituted amide, and has high flexibility because of less steric hindrance around the alpha carbon. Therefore, by using a polysarcosine chain as a constituent unit, a hydrophilic block chain having both high hydrophilicity and flexibility is formed.

If the number of sarcosine units in the hydrophilic block chain is 20 or more, the hydrophilic blocks of the adjacent block polymers are easily aggregated with each other, so that a gel is easily formed in the presence of water as a dispersion medium. The upper limit of the number of sarcosine units in the hydrophilic block chain is not particularly limited. The number of sarcosine units in the hydrophilic block chain is preferably 300 or less from the viewpoint of aggregating the hydrophobic blocks of the adjacent polymers to stabilize the gel structure. The number of sarcosine units is more preferably 25 to 200, and still more preferably 30 to 100.

In the hydrophilic block chain, all the sarcosine units may be continuous, or the sarcosine units may be discontinuous as long as the characteristics of polysarcosine described above are not impaired. When the hydrophilic block chain has a monomer unit other than sarcosine, the monomer unit other than sarcosine is not particularly limited, and examples thereof include hydrophilic amino acids or amino acid derivatives. The amino acids include α-amino acids, β-amino acids and γ-amino acids, and are preferably α-amino acids. Examples of hydrophilic α-amino acids include serine, threonine, lysine, aspartic acid and glutamic acid. In addition, the hydrophilic block may have a sugar chain, a polyether or the like. The hydrophilic block preferably has a hydrophilic group such as a hydroxyl group at the end (the end opposite to the linker with the hydrophobic block).

(Structure and Synthesis Method of Amphiphilic Block Polymer)

The amphiphilic block polymer is obtained by binding a hydrophilic block chain and a hydrophobic block chain. The hydrophilic block chain and the hydrophobic block chain may be bound via a linker. As the linker, those having a lactic acid monomer (lactic acid or lactide) which is a constituent unit of the hydrophobic block chain or a functional group (for example, a hydroxyl group or an amino group) capable of binding to a polylactic acid chain and a sarcosine monomer (for example, sarcosine or N-carboxysarcosine anhydride) which is a constituent unit of the hydrophilic block or a functional group (for example, an amino group) capable of binding to polysarcosine are preferably used. By selecting the linker appropriately, the branched structures of the hydrophilic block chain and the hydrophobic block chain can be controlled.

The method for synthesizing the amphiphilic block polymer is not particularly limited, and known peptide synthesis methods, polyester synthesis methods, depsipeptide synthesis methods and the like can be used. In particular, amphiphilic block polymers can be synthesized with reference to WO 2009/148121 and the like.

In order to adjust gel stability and biodegradability, it is preferred to adjust the chain length of polylactic acid in the hydrophobic block chain and the chain length ratio of the hydrophobic block chain to the hydrophilic block chain (ratio of the number of lactic acid units to the number of sarcosine units). In order to facilitate control of the chain length of polylactic acid, it is preferable to first synthesize polylactic acid having a linker introduced at one end and then to introduce polysarcosine, in the synthesis of the amphiphilic block polymer. The chain lengths of the polysarcosine chain and the polylactic acid chain can be adjusted by adjusting the conditions such as the charging ratio between the initiator and the monomer in the polymerization reaction, the reaction time, and the temperature. The chain lengths of the hydrophilic block chain and the hydrophobic block chain (molecular weight of the amphiphilic block polymer) can be confirmed, for example, by ¹H-NMR. From the viewpoint of enhancing the biodegradability of the amphiphilic block polymer, the weight average molecular weight is preferably 10000 or less, more preferably 9000 or less. The amphiphilic block polymer used in the present invention may form chemical crosslinks between molecules for the purpose of promoting gel formation, improving the stability of the gel, and the like.

[Dispersion Medium]

An aqueous liquid is used as a dispersion medium of the hydrogel. The aqueous liquid is water or an aqueous solution. As the aqueous solution, biochemically and pharmaceutically acceptable aqueous solutions such as distilled water for injection, physiological saline and buffer are preferably used.

From the viewpoint of reducing toxicity and irritation to a living body, it is preferable that the aqueous liquid as the dispersion medium do not substantially contain an organic solvent. The organic solvent content of the dispersion medium is preferably 0.1% by weight or less, more preferably 0.05% by weight or less, and still more preferably 0.01% by weight or less. It is preferable that the dispersion medium be substantially free of an organic solvent also from the viewpoint of promoting the degradation of the gel due to the concentration change and imparting immediate degradability.

[Preparation of Hydrogel Composition]

A hydrogel composition is obtained by mixing the amphiphilic block polymer with the aqueous liquid. As a method of mixing the amphiphilic block polymer and the aqueous liquid, there are indicated: a method of dissolving or swelling the amphiphilic polymer in an organic solvent to form a solution or an organogel, and then replacing the organic solvent with the aqueous liquid; and a method of swelling the solid (powdery) amphiphilic block polymer in the aqueous liquid. In the former method, it is easy to obtain a hydrogel in which the amphiphilic block polymer forms a rod-like molecular assembly. In the latter method, it is easy to obtain a hydrogel in which the amphiphilic block polymer forms a particulate molecular assembly (nanoparticles).

In the hydrogel (hydrogel nano-particles) formed of the nanoparticles, the outer peripheries of the nanoparticles are linked by physical crosslinking to form a gel. As described above, in the amphiphilic block polymer, the hydrophobic block chains easily aggregate in the presence of a dispersion medium. When the hydrophobic block chains aggregate to form a core, the hydrophilic block chains are directed outward, and the molecules self-assemble to form spherical micelles. It is considered that, if water as a dispersion medium is present on the outer peripheries of the micelles, the micelles form a physical crosslink by hydrophilic interaction of the hydrophilic block chains of the adjacent micelles and the water as a dispersion medium to constitute a gel.

When the amphiphilic block polymer is present as an aggregate of nanoparticles in the hydrogel, the particle diameter is preferably 100 nm or less, more preferably 5 to 70 nm, and still more preferably 10 to 50 nm. The particle diameter of the nanoparticles can be adjusted by the composition and molecular weight of the amphiphilic block polymer, the ratio of the amphiphilic block polymer to the dispersion medium, and the like.

When the amount of water as the dispersion medium increases, the distance between the adjacent micelles increases. The physical crosslinking ability due to the hydrophilic interaction between the micelles is weakened, and the gel structure disappears, resulting in conversion to nanoparticles or string-like structures. Therefore, when the hydrogel composition of the present invention is administered into a living body, the gel structure disappears in a short time due to the influence of moisture in the living body. The hydrogel composition of the present invention preferably loses its gel structure within 24 hours after contact with water.

The ratio of the amphiphilic block polymer to water in the hydrogel is not particularly limited, and may be set in a range in which the polymer can be wetted, depending on the molecular weight, mass and the like of the amphiphilic block polymer. When the hydrogel is introduced into a living body by injection, the amount of water may be adjusted so that the hydrogel has an injectable viscosity range.

From the viewpoint of maintaining the gel state by physical crosslinking, the amount of water in the hydrogel composition is preferably 4000 parts by weight or less, more preferably 2000 parts by weight or less, still more preferably 1000 parts or less, particularly preferably 500 parts by weight or less, based on 100 parts by weight of the amphiphilic block polymer. From the viewpoint of preparing a hydrogel having a viscosity suitable for promoting formation of hydrogel particles by polymer wetting and for introduction into the body by injection or the like, the amount of water in the hydrogel composition is preferably 100 parts by weight or more, more preferably 150 parts by weight or more, and still more preferably 200 parts by weight or more, based on 100 parts by weight of the amphiphilic block polymer.

A xerogel may be formed by swelling the amphiphilic block polymer in the solid state in water to form a hydrogel and then removing water. The hydrogel is obtained by swelling the xerogel in water again.

<Other Components Constituting Composition>

The hydrogel composition of the present invention may contain additional components other than the amphiphilic block polymer and dispersion medium described above. For example, the hydrogel composition can include an agent as an additional component. The drug is not particularly limited as long as it acts on a living body and is physiologically acceptable, and examples thereof include anti-inflammatory agents, analgesics, antibiotics, cell cycle inhibitors, local anesthetics, vascular endothelial cell growth factors, immunosuppressants, chemotherapeutic agents, steroids, hormones, growth factors, psychotropic drugs, anticancer drugs, angiogenesis drugs, angiogenesis inhibitors, antiviral drugs, ophthalmic drugs, proteins (enzymes, antibodies, etc.), and nucleic acids. In addition, by inclusion of a signal agent such as a fluorescent labeling agent as the drug, applications as a probe for biological imaging such as fluorescence imaging, ultrasonic imaging, photoacoustic imaging, etc. can also be expected. The hydrogel composition may contain additional components other than the drug, such as preservatives, plasticizers, surfactants, antifoaming agents, stabilizers, buffers, pH regulators, osmotic pressure regulators, and tonicity agents.

The additional components described above may be added at any stage of hydrogel composition preparation. For example, the additional components may be included in the aqueous liquid as the dispersion medium, and the additional components may be added when mixing the amphiphilic block polymer and the aqueous liquid. Also, after preparing the hydrogel, the additional components may be added and mixed.

The hydrogel composition preferably contains as little organic solvent as possible. As described above, in the method of swelling the solid amphiphilic block polymer in the aqueous liquid, a hydrogel composition substantially free of an organic solvent can be obtained because no organic solvent is used for preparation of the hydrogel composition. In addition, the residual organic solvent used at the time of synthesis of the amphiphilic polymer is permissible even if contained in the hydrogel composition. The content of the organic solvent in the hydrogel composition is preferably 0.1% by weight or less, and more preferably 0.05% by weight or less. Among the organic solvents, a lower alcohol having 1 to 6 carbon atoms easily causes inflammation in a living body, so the content of the lower alcohol in the hydrogel composition is preferably 0.01% by weight or less, more preferably 0.005% by weight or less, and still more preferably 0.0001% by weight or less. If no lower alcohol is used in the synthesis of the amphiphilic polymer, an alcohol-free hydrogel composition substantially free of alcohols can be prepared.

[Application of Hydrogel Composition]

The hydrogel composition of the present invention can be administered into a living body for the purpose of treatment or examination. The living body to be administered may be a human or a non-human animal. The method of administering the hydrogel composition to a living body is not particularly limited. Examples of the administering method include transmucosal, oral, instillation, transdermal, nasal, intramuscular, subcutaneous, intraperitoneal, intraarticular, intraocular, intralocular, intramural, intraoperative, intraparietal, intraperitoneal, intrapleural, intrapulmonary, intrathecal, intrathoracic, intratracheal, intratympanic, and intrauterine.

The hydrogel composition of the present invention maintains a gel state prior to administration to a living body, and when administered into a living body, the gel structure disappears in a short time upon contact with a body fluid. Therefore, if the substance to be administered is administered into a living body in a state of being contained in or mixed with the hydrogel composition, the substance to be administered can be made to act in the living body in a short time.

In addition to the above-mentioned various drugs, cells for transplantation can also be used as the substance to be administered. That is, the hydrogel composition of the present invention can also be used as a carrier substrate for cell transplantation. In cell transplantation into a living body, the engraftment rate is low when cells are dispersed. Therefore, in order to improve the engraftment rate, it is desirable to introduce a plurality of cells fixed on the carrier substrate into the living body. The hydrogel composition of the present invention can be administered into a living body in a supported state without dispersing a plurality of cells. After administration into the living body, the gel structure disappears in a short time, so the hydrogen composition hardly causes inhibition of the engraftment of cells. Moreover, since the composition is substantially free of an organic solvent such as alcohols, the inflammation at the transplantation site can be suppressed, so that the load to the living body can be reduced.

The hydrogel composition of the present invention can be used as a filler or the like even when it does not contain drugs, cells or the like. The hydrogel composition of the present invention can be expected not only to be used in pharmaceutical applications, but also to be applied in the fields of cosmetics, foods, agriculture and the like.

### EXAMPLES

Hereinafter, the present invention will be described in more detail by way of examples, but the present invention is not limited to these examples.

### Synthesis Example

### Synthesis of Amphiphilic Block Polymer

With reference to the method described in WO2009/148121, sarcosine anhydride and aminated poly-L-lactic acid were used as monomer components, and glycolic acid, O-(benzotriazol-1-yl)-N,N,N′,N′-tetramethyluronium hexafluorophosphate (HATU) and N,N-diisopropylethylamine (DIEA) were used to synthesize a linear amphiphilic block polymer (PLA₃₂-PSar₁₀₈) having a hydrophilic block consisting of 108 sarcosine units and a hydrophobic block consisting of 32 L-lactic acid units.

### Example 1

### Preparation of Hydrogel

### Preparation Example 1

### Example

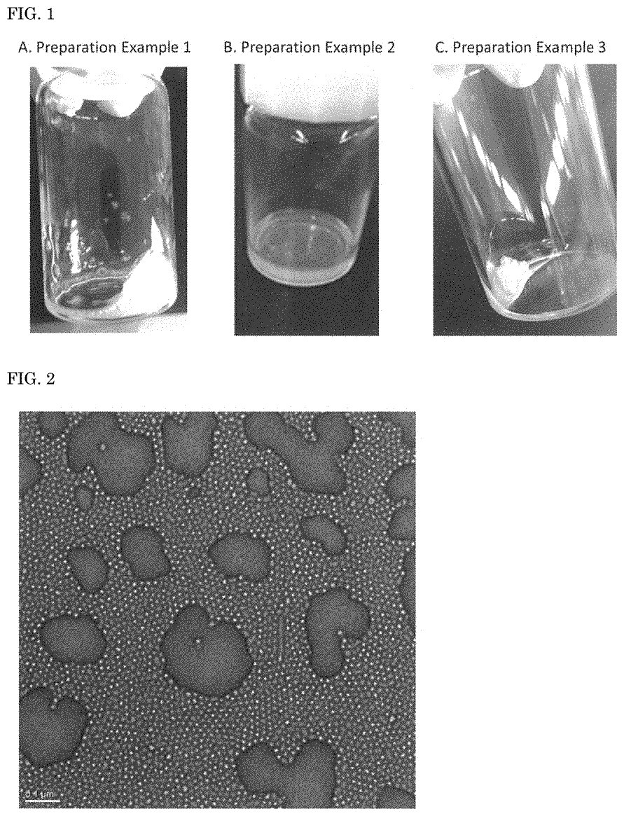

To 100 mg of powder of the polymer obtained in the Synthesis Example, 400 μL of distilled water was added, and they were mixed with a spatula to swell the polymer, thereby obtaining a white wet gel having no fluidity (FIG. 1A).

### Preparation Example 2

### Reference Example

To 200 mg of powder of the polymer obtained in the Synthesis Example, 1000 μL of ethanol was added, and the mixture was heated to 70° C. to dissolve the polymer, thereby obtaining a milky white solution. The solution was cooled at 4° C. for 1 hour to obtain a white wet gel having no fluidity. The organogel was set in a desiccator and dried under reduced pressure overnight (about 12 hours) to obtain a dried gel product (xerogel) from which the solvent was removed. When 1000 μL of distilled water was added to this xerogel and the mixture was allowed to stand at room temperature for 4 hours, the gel became wet and a milky white hydrogel having fluidity was obtained (FIG. 1B).

### Preparation Example 3

### Comparative Example

When 400 μL of distilled water was added to 100 mg of PLGA having a weight average molecular weight of about 5000 (a random copolymer of L-lactic acid and glycolic acid in a molar ratio of 1:1; PLGA 5005 made by Wako Pure Chemical Industries, Ltd.) and they were mixed with a spatula, the polymer did not dissolve or swell, and was completely separated into the solid phase of the polymer and the liquid phase of water (FIG. 1C).

### Preparation Example 4

### Comparative Example

To 100 mg of PLGA having a weight average molecular weight of about 5000, 122.5 μL of N-methylpyrrolidone and 277.5 μL of distilled water were added, and they were mixed with a spatula to swell the polymer, thereby obtaining a gel having viscosity.

(Microscopic Observation of Hydrogel)

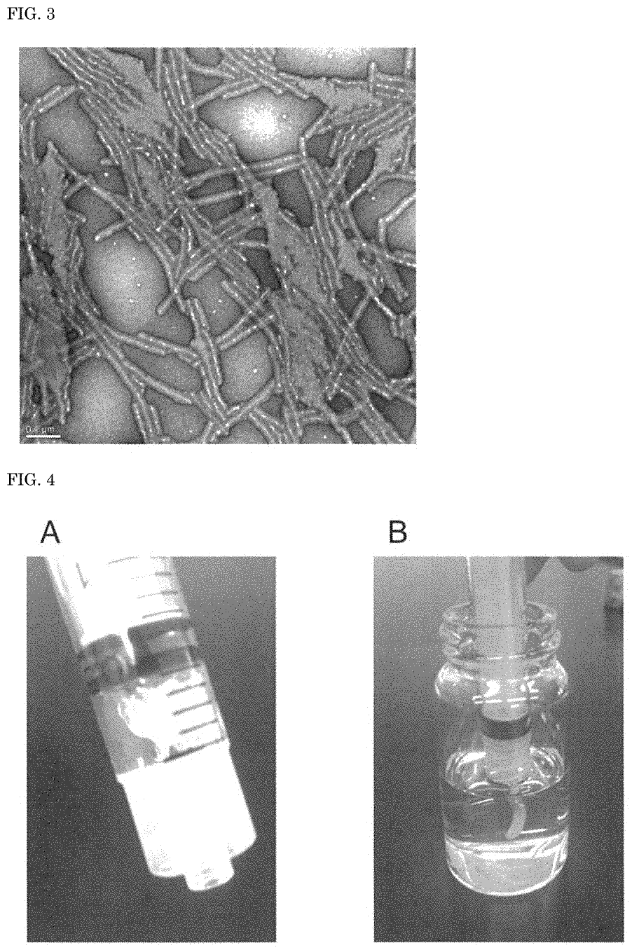

In order to confirm the microstructure of the hydrogels obtained in Preparation Examples 1 and 2, the gels were dried to remove moisture, and observation was then performed with a transmission electron microscope (TEM). FIG. 2 is a TEM observation image of the hydrogel of Preparation Example 1, and a structure in which nanoparticles having a particle diameter of about 10 to 30 nm were connected at the outer peripheral portion was observed. FIG. 3 is a TEM observation image of the hydrogel of Preparation Example 2, and a rod-like structure in which molecular assemblies having a width of about 30 to 50 nm and a length of about 1 to 3 μm were stacked was observed.

The hydrogels of Preparation Examples 1 and 2 were made of the same polymer as a raw material, but a clear difference was confirmed between the microscopic structures of the hydrogels. An organogel made from a solution in which the polymer is dissolved in an organic solvent tends to have a structure in which the hydrophobic block portion of the amphiphilic polymer is aggregated, so large molecular assemblies are considered to be easily formed as compared with the case of a hydrogel in which the polymer is directly swollen in water. In the case of preparing a hydrogel from a xerogel from which the dispersion medium (organic solvent) of the organogel has been removed as in Preparation Example 2, water easily penetrates into the hydrophilic block chain portion of the amphiphilic polymer. Thus, a hydrogel maintaining the same polymer matrix structure as the organogel is considered to have been formed in Preparation Example 2. On the other hand, it is considered that, in Preparation Example 1, aggregation of the hydrophilic block chains is likely to occur subsequently to the aggregation of the hydrophobic block chains because of the presence of water as a dispersion medium, resulting in formation of a hydrogel in which spherical micelles are connected by physical crosslinking.

### Example 2

### Degradability Confirmation Test of Hydrogel

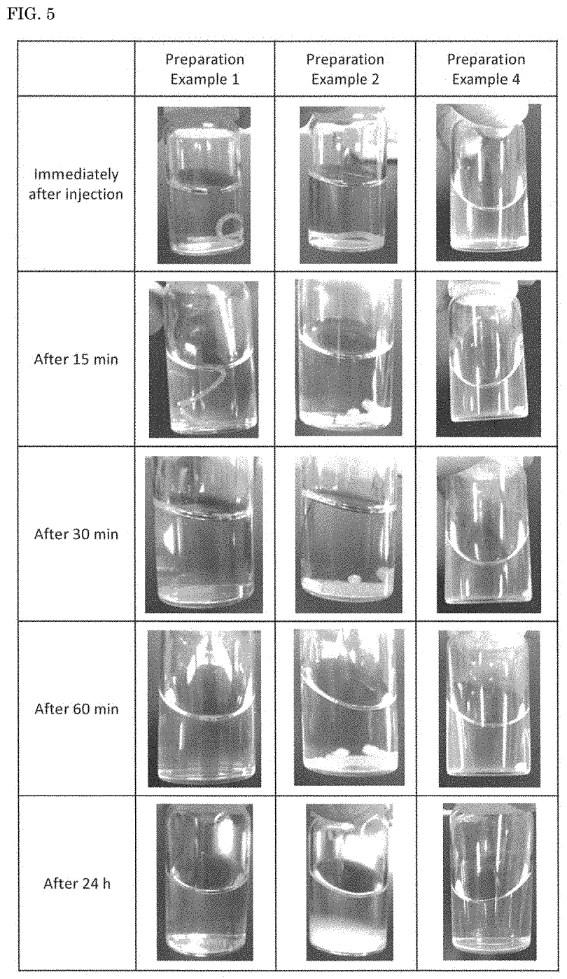

Into a 2.5 mL volume luer lock syringe, 200 mg of the hydrogels obtained in Preparation Examples 1, 2 and 4 were each loaded (FIG. 4A). The hydrogel in the syringe was injected into a vial containing 10 mL of distilled water (FIG. 4B). Thereafter, the vial was allowed to stand, and photographs were taken immediately, 15 minutes, 30 minutes, 60 minutes and 24 hours after injection of the hydrogel to observe changes in the shape of the hydrogel. Appearance photographs are shown in FIG. 5.

As for the hydrogel of Preparation Example 2, a white gel was observed even 60 minutes after injection, and a gel-like precipitate was observed at the bottom of the vial even after 24 hours. As for the hydrogel of Preparation Example 1, on the other hand, the gel became thin in 15 minutes after injection, and most of the gel disappeared after 30 minutes. The PLGA gel of Preparation Example 4 was dispersed in a whitish state immediately after injection into water, and did not keep the form as a gel, and some solid remained in water. This solid remained as a solid even 24 hours after injection.

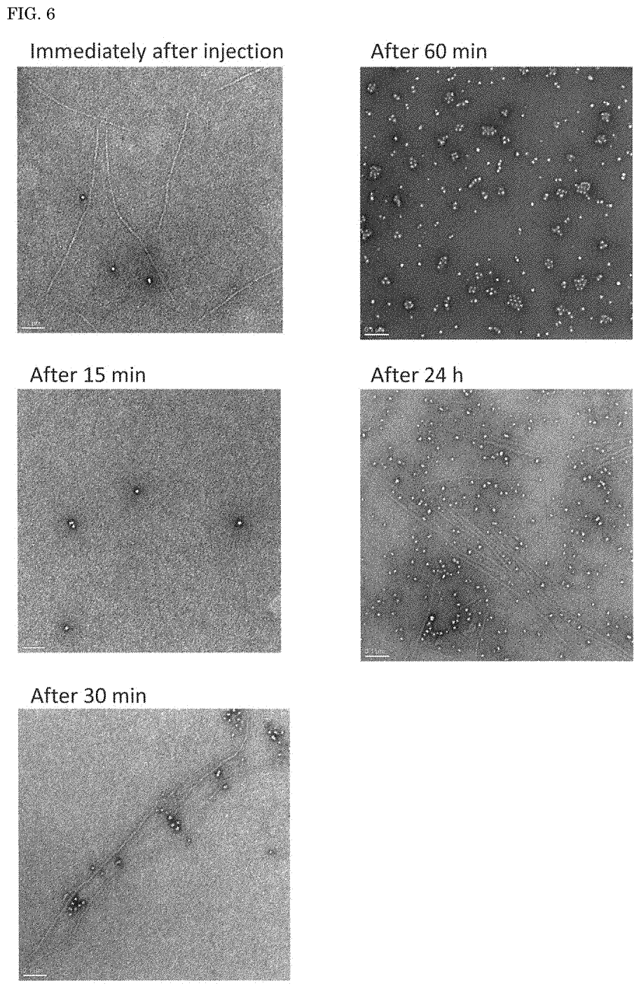

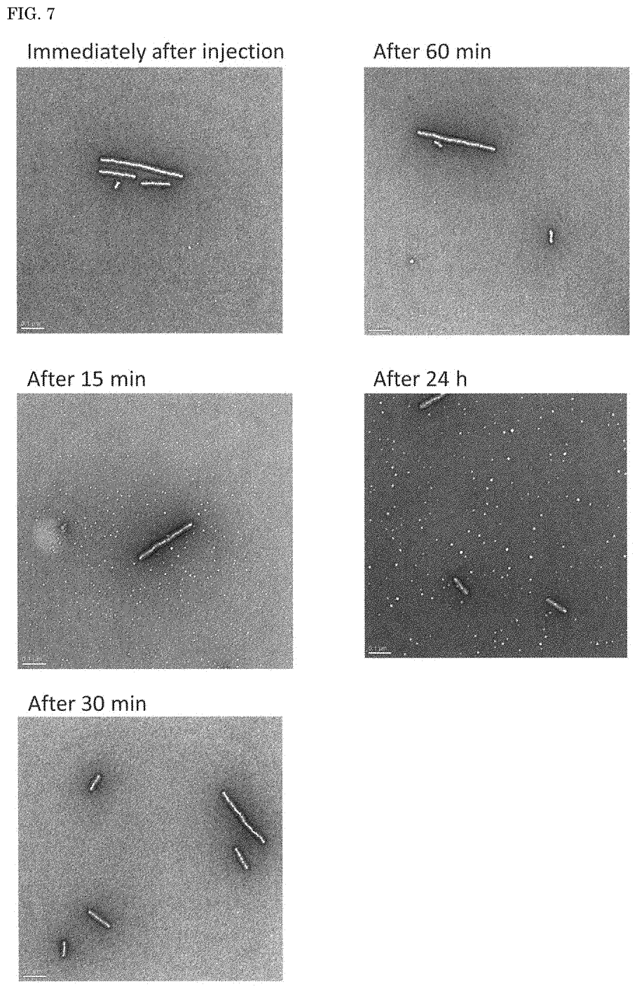

Regarding the hydrogels of Preparation Examples 1 and 2, water in the vial was recovered at the time of photographing (immediate, 15 minutes, 30 minutes, 60 minutes and 24 hours after injection of the gel) to perform TEM observation. TEM observation images of the water recovered from the vials into which the gels of Preparation Examples 1 and 2 were injected are shown in FIGS. 6 and 7, respectively.

In Preparation Example 2 (FIG. 7), rod-like structures were observed immediately after injection of the gel, and almost no nanoparticles were observed. Even after elapse of 15 to 60 minutes, rod-like structures were observed. After elapse of 24 hours, in addition to the rod-like structures, nanoparticles having a particle diameter of about 10 to 20 nm were observed.

In Preparation Example 1 (FIG. 6), the state was observed that nanoparticles of 10 to 30 nm in particle diameter were dispersed in water immediately after gel injection, and the state in which the number of particles dispersed in water increased with the elapse of time was observed. After elapse of 24 hours, in addition to the nanoparticles, string-like structures having a width of about 10 nm were also observed. Combining the results of FIGS. 5 and 6, it can be seen that, in the hydrogel of Preparation Example 1, immediately after injection into water, the gel structure collapses due to the decrease in interaction between the nanoparticles constituting the gel, and that the nanoparticles are released into water.

From the above results, it was seen that the gel of Preparation Example 1 loses its gel structure in a short time and the residence time thereof in a living body is short, as compared with the gel of Preparation Example 2.

1-5. (canceled)

6. A hydrogel composition comprising:
an amphiphilic block polymer, the block polymer including (i) a hydrophilic block chain having a plurality of sarcosine units and (ii) a hydrophobic block chain having a plurality of lactic acid units; and
water added as a dispersion medium,
wherein the hydrogel composition has a rod-like structure overlapped with each other in which molecular assemblies are stacked.

7. The hydrogel composition according to claim 6, wherein the plurality of sarcosine units are 20 or more sarcosine units and the plurality of lactic acid units are 10 or more lactic acid units.

8. The hydrogel composition according to claim 6, wherein the amphiphilic block polymer has the form of hydrogel composed of nano-particles, the nano-particles having a particle diameter of 100 nm or less.

9. The hydrogel composition according to claim 6, wherein a content of an organic solvent is 0.1% by weight or less.

10. The hydrogel composition according to claim 6, wherein a content of a lower alcohol is 0.01% by weight or less.

11. A method for producing a hydrogel composition having a rod-like structure overlapped with each other in which molecular assemblies are stacked, the method comprising:
dissolving an amphiphilic block polymer, the block polymer including (i) a hydrophilic block chain having a plurality of sarcosine units and (ii) a hydrophobic block chain having a plurality of lactic acid units, in an organic solvent to form a solution, or
swelling the amphiphilic block polymer in an organic solvent to form an organogel; and
replacing the organic solvent with an aqueous liquid.

12. A method for producing a particulate hydrogel composition, the method comprising:
preparing a solid amphiphilic block polymer having a hydrophilic block chain having a plurality of sarcosine units and a hydrophobic block chain having a plurality of lactic acid units; and
swelling the solid amphiphilic block polymer in an aqueous liquid.
